(19) United States
(12) Patent Application Publication
(10) Pub. No.: US 2006/0040878 A1
(43) Pub. Date: Feb. 23, 2006

(54) Bacterial antigen induced bone morphogenesis

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 10/922,174

(22) Filed: Aug. 20, 2004

(51) Int. Cl. A61K 48/00; A61K 39/02

(52) U.S. Cl. 514/44; 424/200.1; 424/423

(57) ABSTRACT

Bone growth following a spinal fusion procedure is enhanced by packing the fusion site with a mixture of a bone material such as allograft or autograft, and an antigen produced from bacteria or parasitic organisms. A composition for inducing bone morphogenesis also is disclosed.

### BACKGROUND OF THE INVENTION

This invention relates to a method of,inducing bone morphogenesis using bacterial antigens.

While bacterial infection following a surgical bony fusion procedure of the spine is an extremely undesirable complication, it is well recognized that such infections once controlled with antibiotic therapy frequently go on to develop solid bony fusion with an exuberant growth of bone. The amount of bone development frequently exceeds that seen with surgical fusions not complicated by infection. Indeed, ectopic calcification frequently is seen in resolved infections in many organ systems in the human body, even where bone not is normally present. It appears that bacterial or even parasitic infections have the capacity to enhance bone growth, or even to cause de novo bone formation even in the absence of osteoblasts.

Infection, in medical terms, refers to a host parasite interaction, which encompasses not only the process by which the parasite or pathogen gains a portal of entry into the human body, but also the process by which the body mounts a defensive assault to halt the invasion and propagation of the invading organisms or pathogens.

Part of the defensive assault mounted by the host body is the activation of leukocytes and phagocytic monocytes, which directly attack the invading pathogen or invoke secondary defense processes by releasing substances, such as interieukin I, interleukin II and lymphokines. These substances serve a variety of functions best categorized as immunomodulatory. The inflammatory processes incited by the host-parasite interaction, with or without these immunomodulators, appear to be able to induce or enhance bone morphogenesis.

For a host-parasite interaction to occur, the host must identify the invader as foreign. To do this, host cells recognize various cell surface components as stereo-chemically different from components on the surface of host cells. These identifiable surface characters are variable in their content, configurations and chemical make-up and broadly are referred to as antigens. All bacterial and parasitic organisms contain surface and interior components, which can function as antigens, hence are included as potential instigators of pathogen induced bone morphogenesis.

Of organisms found to cause bone infections, gram positive cocci, such as Staphyloco aureus, are the most common. However, gram negative rods are frequent as well. Regardless, many organisms may infect human osseous tissue and trigger an osteoblastic response. In reality, any pathogen could be used to incite or stimulate a host parasite interaction capable of triggering or fostering bone morphogenesis.

The gram positive genera, which are of prime interest in serving as inducers of bone morphogenesis, include all species of Staphylococcus, Streptococcus, Cornybacterium, Listeria, Erysipelothrix, Bacillus and Clostridium. Gram negative cocci of interest include the genera of Neisseria, Sperillium, Pasturella, Brucella, Yersinia, Francisella, Hemophylus, Bordetella and Legionella. Gram negative rods include Escherichia, Salmonella, Shigella, Klebsiella, Proteus, Vibrio, Pseudomonas, Bacteroides, Fusobacterium and Mycoplasma.

Antigens are component parts of a pathogen capable of instigating a host-parasite interaction. Most gram positive cell walls contain considerable amounts of teichoic and teichuronic acids, which form up to fifty percent of the dry weight of the cell wall and ten percent of the dry weight of the total cell. Teichoic and teichuronic acids are water soluble polymers containing ribitol or glycerol joined via phosphodiester linkages. One main type of teichoic acids is covalently linked to peptidoglycans. The other main type of teichoic acids is covalently linked to membrane glycolipids. The teichoic acids constitute major surface antigens of many of the gram positive bacteria. These antigens in turn are expressed on the cell surface and interact with antibodies of the human immune system as part of the molecular basis of host-parasite interaction.

Some gram positive cell walls contain polysaccharide molecules. These polysaccharide moieties also may play a role in antigenicity.

Gram negative cell walls contain three main components: lipoproteins, lipopolysaccharides and membrane components. Lipoproteins are a combination of lipid and protein structures and are the most abundant protein of gram negative cells. Lipopolysaccharides are a combination of a complex lipid linked to a complex sugar or polysaccharide.

Polysaccharides are present in all gram negative species.

Lipopolysaccharides are believed to be extremely potent antigens. The outer membrane component consists of a phospholipid bilayer in which is embedded various protein molecules. While the embedded proteins are more likely to play a major role in antigenicity, fractionated membranes containing phospholipids may play a role in antigenicity when released in significant amounts from dead or dying bacterial or parasitic cells.

In addition to these components, many bacterial cells synthesize an extracellular polymer, which is either a condensed well-defined capsule or a loose fuzzy mesh called a glycocalyx. This glycocalyx is a type of polysaccharide, which appears to aid in attaching to host cells. Because of its association with the outer surface of the bacterial cell, it may well funcion as a potent antigen.

Finally, many bacteria produce toxins as part of their pathologic response. These toxins are categorized as exotoxins, excreted products, and as endotoxins, part of the organism itself. These toxins also may be capable of serving as antigens and inducing or facilitating bone morphogenesis.

### SUMMARY OF THE INVENTION

These observations suggest that some component of an invading pathogen, a bacterium or parasite, is capable of inducing or enhancing bone morphogenesis. While the exact mechanism by which this occurs remains unclear, it seems reasonable that a suitable bacterium, parasite or other similar organism could be exploited toward this end by using it whole, in a dead or weakened state, or by using a component of its cellular parts (antigen) to induce bone growth or enhance bone growth in the pursuit of a clinically stable bony arthrodesis or fusion. It is also possible that secondary messengers, such as interleukin I, interleukin II, lymphokines, or endogenous pyrogens or prostaglandins may be utilized in inducing or enhancing bone morphogenesis.

The organisms, which most likely may be exploited to enhance bone formation include those known to infect the spine either pathologically or post-surgically, or organisms known to have a propensity to induce ectopic calcification. These organisms could include the various forms of staphylococcus, streptococcus, Escherichia Coli, mycobacterium, and the like, as indicated above.

### DESCRIPTION OF THE PREFERRED EMBODIMENTS

As used herein, allograft bone is obtained from human cadavers and can represent mineralized or demineralized preparations. These may be whole or component parts of human bone or morselized components thereof. Demineralized configurations can be constituted into a gel, paste or putty-type consistency, which more readily lends to mixing with antigen powders or solutions. Mineralized components may have an antigen painted on surfaces thereof so as to biologically inoculate or activate them in this fashion. The antigen also may be sprayed.

Xenograft bone, or bone obtained from other species, e.g. bovine bone, porcine bone, coral, and the like, also are used, as bone morphogenesis induced by antigen inoculation may well override any inherent tendency of the host body to reject cross-species grafting. Demineralized configurations, as described above, also are used.

Autograft bone is available and obtained through harvest of marrow surgically, or through bone marrow aspiration and grafted after admixing with an antigen or secondary messengers, such as interleukin I, II, lymphokines, or endogenous pyrogens and prostaglandins. Demineralized configurations, as described above, also are used.

The following examples illustrate, but do not limit the invention.

### EXAMPLE I

A matrix useful for promoting the growth of bone can be prepared from either allograft, autograft or xenograft bone components. These may include solids, putty or combinations thereof. In addition, demineralized bone treated by agents that demineralize without substantially denaturing the normal collagen matrix of bone may be used. A matrix also may be made from calcium hydroxyapatite, tricalcium phosphate or calcium silicates. Powder or beads of ceramic or glass also may serve as a suitable matrix. Any biologically inert matrix having a particular size sufficient to prevent engulfment by macrophages, i.e. greater than microns, might be used, as it is believed that bone formation requires the infiltration of macrophages into the matrix.

To this chosen matrix, weakened or dead bacterial or parasitic organisms, or components or combinations thereof, are added to the matrix to form a composition to be used as graft material for clinical fusion. The bacteria or parasites may be killed or weakened by chemical, thermal, electrolytic or electromagnetic means, e.g. ultraviolet or gamma radiation, after having been grown in sufficient quantities in various commercially available culture mediums.

In preferred embodiments, dead or weakened bacteria parasitic organisms are added to the chosen bone matrix in a range of about 0.01% to about 0.5% dry weight. The exact amounts may be varied to exceed the above recommendations depending on the clinical response desired. Additional bacterial or parasitic material could be injected into the matrix graft side at a later time if so desired.

### EXAMPLE II

In the same manner as described in Example I, the composition for clinical surgical fusion may contain a crude bacterial lysate made from dead or weakened bacteria lysed through chemical, mechanical, ultrasonic or centrifugal means. This lysate contains components of the cell wall, cytoplasm and nuclear materials, such as DNA and RNA. If crude bacterial lysate is used, the preferred embodiment includes one nanogram to one milligram of crude bacterial lysate for each milligram of bone matrix.

### EXAMPLE III

In the same manner as described in Example I, the composition for clinical surgical fusion may contain a fractionated component of a bacterial cell wall containing teichoic and or teichuronic acid obtained by centrifugation, microfiltration or chromatographic isolation. The preferred embodiment in this configuration includes one nanogram to one milligram of teichoic or teichuronic acid for each milligram of bone matrix.

### EXAMPLE IV

In the same manner as described in Example I, the composition for clinical surgical fusion may contain membrane components obtained by centrifugation, microfiltration or chromatographic isolation. The preferred embodiment in this configuration includes one nanogram to one milligram of membrane components for each milligram of bone matrix.

### EXAMPLE V

In the same manner as described in Example I, the composition for clinical surgical fusion may contain lipoproteins obtained by centrifugation, microfiltration or chromatographic isolation. The preferred embodiment in this configuration includes one nanogram to one milligram of lipoproteins for each milligram of bone matrix.

### EXAMPLE VI

In the same manner as described in Example I, the composition for clinical surgical fusion may contain lipopolysaccharides obtained by centrifugation, microfiltration or chromatographic isolation. The preferred embodiment in this configuration includes one nanogram to one milligram of lipopolysaccharides for each milligram of bone matrix.

### EXAMPLE VII

In the same manner as described in Example I, the composition for clinical surgical fusion may contain glycocalyx obtained by centrifugation, microfiltration or chromatographic isolation. The preferred embodiment in this configuration includes one nanogram to one milligram of glycocalyx for each milligram of bone matrix.

### EXAMPLE VIII

In the same manner as described in Example I, the composition may contain interleukin I isolated from infected or non infected human wound fluid, purified, cloned and recombinantly expressed in a host system, such as a cultured bacteria. The preferred embodiment includes one nanogram to one milligram of interleukin I for each milligram of bone matrix.

### EXAMPLE IX

In the same manner as described in Example I, the composition may contain lymphokines isolated from infected or non infected human wound fluid, purified, cloned and recombinantly expressed in a host system, such as a cultured bacteria. The preferred embodiment includes one nanogram to one milligram of lymphokines for each milligram of bone matrix.

### EXAMPLE X

In the same manner as described in Example I, the composition may contain prostaglandins isolated from infected or non infected human wound fluid, purified, cloned and recombinantly expressed in a host system, such as a cultured bacteria. The preferred embodiment includes one nanogram to one milligram of prostaglandins for each milligram of bone matrix.

### EXAMPLE XI

In the same manner as described in Example I, the composition may contain C reactive protein isolated from infected or non infected human wound fluid, purified, cloned and recombinantly expressed in a host system, such as a cultured bacteria. The preferred embodiment includes one nanogram to one milligram of C reactive protein for each milligram of bone matrix.

### EXAMPLE XII

In the same manner as described in Example I, the composition may contain endogenous pyrogens isolated from infected or non infected human wound fluid, purified, cloned and recombinantly expressed in a host system, such as a cultured bacteria. The preferred embodiment includes one nanogram to one milligram of endogenous pyrogens for each milligram of bone matrix.

Since the invention is subject to modifications and variations, it is intended that the foregoing description shall be interpreted as only illustrative of the invention defined by the following claims.

I claim:

1. Method of inducing bone morphogenesis comprising introducing into a surgical site a composition of bone material or matrix and an antigen.

2. Method of claim 1, wherein the surgical site is between bones selected to be surgically fused in an animal or human being.

3. Method of claim 1, wherein the surgical site is between vertebrae of a spinal column.

4. Method of claim 1, wherein the bone material is selected from the group consisting of allograft material, autograft material, xenograft material, a calcium collagen admixture and combinations thereof.

5. Method of claim 1, wherein the bone material is demineralized.

6. Method of claim 1, wherein the bone matrix comprises particles larger than a microphage or 75 microns.

7. Method of claim 1, wherein the bone matrix comprises ceramic powder or beads, glass powder or beads, metal powder or beads, calcium hydroxyapatite, calcium silicate, tricalcium phosphate or combinations thereof.

8. Method of claim 1, wherein the antigen is selected from the group consisting of organisms known to infect the spine either pathologically or post-surgically, organisms known to have a propensity to induce ectopic calcification, and combinations thereof.

9. Method of claim 1, wherein the antigen is selected from the group consisting of weakened bacteria, dead bacteria, weakened parasitic organisms, dead parasitic organisms and combinations thereof.

10. Method of claim 9, wherein the composition comprises 0.01% to 0.5% of the antigen by weight.

11. Method of claim 1, wherein the antigen is selected from the group consisting of Staphylococcus, Streptococcus, Cornybacterium, Listeria, Erysipelothrix, Bacillus, Clostridium, Neisseria, Sperillium, Pasturella, Brucella, Yersinia, Francisella, Hemophylus, Bordetella, Legionella, Escherichia, Salmonella, Shigella, Klebsiella, Proteus, Vibrio, Pseudomonas, Bacteroides, Fusobacterium, Mycoplasma, Escherichia Coli, mycobacterium and combinations thereof.

12. Method of claim 1, wherein the antigen comprises a crude bacterial lysate, a teichoic acid, a teichuronic acid, a fractionated component of a bacterial or parasitic cell wall, cytoplasm, membrane, DNA or RNA, a lipoprotein, a lipopolysaccharide, a polysaccharide, a glycocalyx or combinations thereof.

13. Method of claim 12, wherein a proportion of antigen-to-bone material or matrix ranges from one nanogram to one milligram of antigen for each milligram of bone matrix.

14. Method of claim 1, further comprising introducing into the surgical site a chemoattractant for triggering bone morphogenesis.

15. Method of claim 14, wherein the chemoattractant is selected from the group consisting of interleukin I, interleukin II, lymphokine, C reactive protein, endogenous pyrogen, prostaglandin and combinations thereof.

16. Method of claim 15, wherein a proportion of chemoattractant-to-bone material or matrix ranges from one nanogram to one milligram of chemoattractant for each milligram of bone matrix.

17. Composition for inducing bone morphogenesis comprising an effective amount of:
bone material or matrix; and
an antigen.

18. Composition of claim 17, wherein said bone material is selected from the group consisting of allograft material, autograft material, xenograft material, a calcium collagen admixture and combinations thereof.

19. Composition of claim 17, wherein said bone material is demineralized.

20. Composition of claim 17, wherein said bone matrix comprises particles larger than a microphage or 75 microns.

21. Composition of claim 17, wherein said bone matrix comprises ceramic powder or beads, glass powder or beads, metal powder or beads, calcium hydroxyapatite, calcium silicate, tricalcium phosphate or combinations thereof.

22. Composition of claim 17, wherein said antigen is selected from the group consisting of organisms known to infect the spine either pathologically or post-surgically, organisms known to have a propensity to induce ectopic calcification, and combinations thereof.

23. Composition of claim 17, wherein said antigen is selected from the group consisting of weakened bacteria, dead bacteria, weakened parasitic organisms, dead parasitic organisms and combinations thereof.

24. Composition of claim 23, wherein said composition comprises 0.01% to 0.5% of said antigen by weight.

25. Composition of claim 17, wherein said antigen is selected from the group consisting of Staphylococcus, Streptococcus, Cornybacterium, Listeria, Erysipelothrix, Bacillus, Clostridium, Neisseria, Sperillium, Pasturella, Brucella, Yersinia, Francisella, Hemophylus, Bordetella, Legionella, Escherichia, Salmonella, Shigella, Klebsiella, Proteus, Vibrio, Pseudomonas, Bacteroides, Fusobacterium, Mycoplasma, Escherichia Coli, mycobacterium and combinations thereof.

26. Composition of claim 17, wherein said antigen comprises a crude bacterial lysate, a teichoic acid, a teichuronic acid, a fractionated component of a bacterial or parasitic cell wall, membrane, cytoplasm, DNA or RNA, a lipoprotein, a lipopolysaccharide, a polysaccharide, a glycocalyx or combinations thereof.

27. Composition of claim 26, wherein a proportion of antigen-to-bone material or matrix ranges from one nanogram to one milligram of antigen for each milligram of bone matrix.

28. Composition of claim 17, further comprising introducing into the surgical site a chemoattractant for triggering bone morphogenesis.

29. Composition of claim 28, wherein said chemoattractant is selected from the group consisting of interleukin I, interleukin II, lymphokine, C reactive protein, endogenous pyrogen, prostaglandin and combinations thereof.

30. Composition of claim 29, wherein a proportion of chemoattractant-to-bone material or matrix ranges from one nanogram to one milligram of chemoattractant for each milligram of bone matrix.
